(19) United States
(12) Patent Application Publication
(10) Pub. No.: US 2009/0099524 A1
(43) Pub. Date: Apr. 16, 2009

(54) DEVICE FOR ADMINISTERING A FLUID PRODUCT

(76) Inventors: Fritz Kirchhofer, Sumiswald (CH); Jurg Clavadetscher, Ortschwaben (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/270,049

(22) Filed: Nov. 13, 2008

Related U.S. Application Data
(63) Continuation of application No. PCT/CH2006/000258, filed on May 15, 2006.

(51) Int. Cl. A61M 5/20

(52) U.S. Cl. 604/151

(57) ABSTRACT

A device for administering a substance, e.g. a medicament or a therapeutic agent, at a controllable administration rate, wherein the device includes a housing with a drive receptacle area for a drive unit, a substance receiving region to hold a substance container and an arrangement for hydraulic power transmission, which does not contribute to controlling the administration rate, located between the drive receptacle area and the substance receiving region, whereby the administration rate is controlled exclusively by the drive unit.

### CROSS-REFERENCED RELATED APPLICATIONS

This application is a continuation of International Patent Application No. PCT/CH2006/000258 filed May 15, 2006, the entire content of which is incorporated herein by reference.

### BACKGROUND

The present invention relates to devices for administering, delivering, infusing, injecting or dispensing a substance, and to methods of making and using such devices. More particularly, it relates to a device for administering a fluid product or substance, such as a therapeutic or medicinal substance or a medicament.

For various illnesses and physical conditions it may be necessary to administer to a patient a medicament or therapeutic substance in fluid form, e.g. insulin or blood-thinning medicaments such as heparin, continuously and over a relatively long period. Various administering devices are known for this purpose. In particular, infusion devices or infusion pumps are known in which the medicament is contained in an ampoule, e.g. a glass ampoule. The ampoule is placed in the infusion device and connected via a catheter to a cannula which terminates, e.g. subcutaneously, in the body tissue of the patient. A stopper is arranged displaceably in the ampoule and is powered or driven for movement, e.g. via an approppriate gear and/or an electromotor, such that the medicament is dispensed to the patient through the catheter and the cannula.

Such infusion devices may be designed to be as small and flat as possible to be worn inconspicuously on the body of the patient. Yet pumps in which the stopper is driven directly are subject to certain restrictions with respect to possible structural forms, since the drive constituted by motor and gearbox must be in direct connection with the stopper of the ampoule. This limits the design flexibility for such pumps.

In addition, such pumps are typically relatively complex in construction and thus cost-intensive to manufacture. The pumps are therefore not disposable items for one-off use, but are used repeatedly. Whenever an ampoule is emptied, the patient or a caregiver replaces it with a new, full ampoule. With insulin pumps, for example, this happens normally once to several times weekly. Replacing the ampoule is a delicate procedure for a number of reasons. For example, the ampoules generally are made of glass and can shatter when being replaced. Also, the replacement procedure is a relatively complicated procedure, prone to error, for which the patient therefore must be specially trained. In addition, such replacing is not satisfactory from a hygienic standpoint, since movable parts of the pump come into direct contact with the movable stopper of the ampoule, and germs could be introduced into the ampoule in the event of a leak. Similar problems or difficulties may be attributed to manually operated injectors, which aid in administering a preset one-off dose at specific times, because the driving or injection mechanism in such injectors is normally relatively complicated and therefore expensive, exchangeable ampoules are frequently used.

In the prior art comparatively simply designed infusion devices have been proposed, which manage without a motor drive and therefore can be manufactured cost-effectively. In some instances, such devices are powered, e.g. by spring force. In such devices a stopper is usually arranged displaceably in an ampoule. To easily ensure a temporally constant, minimal discharge rate of a medicament in the ampoule, it has been proposed to power the stopper hydraulically and to provide an arrangement for the limiting of throughflow in the hydraulic section, leading to a constant, minimal throughflow rate through the hydraulic section. An example of such a device is described in DE-A 199 39 023, wherein a long capillary of small cross-section is provided in the hydraulic section, the capillary acting to limit throughflow and to reduce pressure.

Another example is disclosed in DE-A 101 02 814. In this case, the medicament is provided in a compressible medicament reservoir. Pressurized hydraulic fluid is provided in a likewise compressible hydraulic reservoir. Via a proportioning device, in particular in the form of a capillary, the fluid enters a shift reservoir, which compresses the medicament reservoir and thus causes the medicament to be discharged.

Such devices exhibit a series of disadvantages. In particular, it is only possible to dispense the medicament at a predetermined, constant rate, determined by the structure and geometry of the proportioning device, in particular by length and cross-section of the capillary section. Control or variation of the administration rate is therefore not possible. In practice, however, it is frequently necessary to regulate the administration rate to adjust it individually to the needs of a patient. In addition, it is frequently desired, in addition to the constant discharge at a small rate, the so-called basal rate, to provide an on-demand increased quantity of the medicament, a so-called bolus. When insulin is administered a bolus may be required, e.g. after meals, when the body has an increased insulin requirement. This is not possible with the known devices with capillaries; normally an additional injection set is required for administering a bolus.

An added drawback is that the capillary section must be manufactured very precisely so as to avoid overly large variations in the discharge rate. This is especially evident from the Hagen-Poiseuille law which sets the throughflow rate through a tubular capillary during laminar flow in relation with the dimensions of the capillary. According to this law, the diameter of the capillary enters the fourth power in calculating the throughflow rate. What are now small variations in the capillary cross-section result in large variations in the throughflow rate. Since the capillary requires highly precise manufacturing, the manufacturing costs are relatively high, which in turn renders such devices only of limited usefulness for one-off use.

### SUMMARY

In one embodiment, the present invention comprises a device for administering a fluid product or substance, which is easy to handle, which enables individual, selective control of the discharge rate and/or discharge quantity of the fluid product, and which can be manufactured cost-effectively, including, in some instances, as a disposable article for one-off use.

In one embodiment, the present invention comprises a device for administering a substance, e.g. a medicament or a therapeutic agent, at a controllable administration rate, wherein the device includes a housing with a drive receptacle area for a drive unit, a substance receiving region to hold a substance container and an arrangement for hydraulic power transmission, which does not contribute to controlling the administration rate, located between the drive receptacle area and the substance receiving region, whereby the administration rate is controlled exclusively by the drive unit.

In one embodiment, the present invention comprises an administering device for administering a fluid product with regulatable administration rate, comprising a housing with a drive-receiving region for a drive unit, a product-receiving region for receiving a product container with the fluid product, and a structure for hydraulic power transmission between the drive-receiving region and the product-receiving region, wherein the structure for hydraulic power transmission has no effect on the administration rate.

In some embodiments, the present invention comprises a system for administering a fluid product comprising an administering device having an associated selectively variable, controllable, regulatable administration rate, the device comprising a housing with a drive-receiving region for a drive unit, a product-receiving region for receiving a product container with the fluid product, and a structure for hydraulic power transmission between the drive-receiving region and the product-receiving region, wherein the structure for hydraulic power transmission makes no contribution to controlling the administration rate, and a drive unit for operable coupling to the drive-receiving region, the drive unit being adapted to supply the structure for hydraulic power transmission with a driving pressure. In some embodiments of the administering device and/or the system, the associated administration rate is controlled exclusively by the drive unit.

In some embodiments, the present iventon comprises a method of manufacturing an administering device device for administering a fluid product at regulatable administration rate, comprising a housing with a drive-receiving region for a drive unit, a product-receiving region for receiving a product container with the fluid product, and a structure for hydraulic power transmission between the drive-receiving region and the product-receiving region, wherein the structure for hydraulic power transmission makes no contribution to controlling the administration rate, the method comprising the steps of providing a housing of the device with an as yet unfilled hydraulic reservoir arranged therein and the product container filled with the fluid product, and subsequently filling the hydraulic reservoir with a hydraulic fluid. In some embodiments, the hydraulic fluid is supplied with excess pressure during filling.

In one embodiment, the present invention comprises an administering device for administering a fluid product, e.g. a medicament in fluid form, e.g. an insulin solution, to a patient, wherein the administration rate of the product can be varied and/or regulated. The device comprises a housing with a drive-receiving region for a drive unit, a product-receiving region for receiving a product container with the fluid product, and a structure for hydraulic power transmission between the drive-receiving region and the product-receiving region. In some embodiments, the structure for hydraulic power transmission is designed such that it makes no contribution to controlling or affecting the administration rate, e.g. the hydraulics are not decisive for the administration rate.

In that, according some embodiments of the present invention, a hydraulic power transmission is provided, this enables considerable flexibility for the delivery device and possible features and/or structures realted thereto. In addition, since the device has no mechanical components requiring highly precise manufacturing, it can be manufactured very cost-effectively and is suited for manufacturing as a disposable article for one-time use. The device can be delivered to the patient with a factory-inserted product containerm whereby the patient needs only to insert the drive unit in the device. This simplifies handling compared to a conventional unit in which the patient has to exchange the actual product container, and improves hygiene. Since the hydraulic section is not determinative for the administration rate and serves only as power transmission, free control of the administration rate is enabled. Changes in the administration rate are therefore possible, without constructive changes having to be made to the device, e.g. during operation of the device.

In one embodiment of the present invention, a structure for hydraulic power transmission in an administration device comprises a hydraulic reservoir with a hydraulic fluid, wherein the hydraulic reservoir is supplied with a driving pressure by a drive unit inserted into a drive-receiving region of the device, and a fluid connection (hydraulic section) between the hydraulic reservoir and a shift reservoir, wherein the fluid connection is designed such that a driving pressure present in the hydraulic reservoir can be transmitted via the fluid connection and the shift reservoir to a fluid product in a product container arranged in a product-receiving region of the device, and wherein the fluid connection has a cross-section large enough and, in some embodiments, large enough everywhere, for the fluid connection to make no contribution to controlling the administration rate. In some preferred embodiments, the device further comprises the product container with the fluid product arranged in the product-receiving region of a housing of the device. In this case, the shift reservoir can be delimited at least partially by the product container. In some embodiments, the shift reservoir could be an independent container. In some preferred embodiments, together with the product container located therein, the device is designed as a disposable article for one-off use.

In one embodiment of the present invention, the product container may take the form of a conventional ampoule. In this case, the product container comprises a rigid, cylindrical, e.g. circular-cylindrical side wall region and a product stopper sealed to be impermeable to fluid and displaceable therein. The stopper is arranged such that it can be displaced by expansion of the shift reservoir. In one embodiment, the shift reservoir is not formed by an independent container, but is delimited at least partially directly by the cylindrical side wall region of the product container and the side of the product stopper away from the fluid product. The product stopper therefore can be said to subdivide the product container into a region which contains the actual product, and a region containing or comprising the shift reservoir.

Alternatively, in some embodiments, the product container can be compressible as a whole, e.g. designed such that the volume of the product container can be altered without parts thereof in contact with the fluid product sliding or moving against one another. Sealing of such parts for this purpose, which would be required and raise manufacturing costs, can thus be omitted, and the form of the product container can be selected extensively freely. This enables improved hygiene, and it is easily possible to adapt the product container in its form and physical dimensions, e.g. its thickness, to special requirements. In some preferred embodiments, such a product container has at least one wall region, the form and/or dimensions of which can be altered such that a change in volume of the product container occurs. A wall region can be designed as bellows, for example. Alternatively, the wall region can be formed, for example, by or from a flexible film. It is also possible to form the wall region from an elastomer material such that a change in volume of the product container occurs with elastic expansion of the material. In some preferred embodiments, the product container may at the same time have at least one, in some preferred embodiments, two, dimensionally stable end regions which can be moved against one another, e.g. in the form of a dimensionally stable end-side terminal wall, which makes it easier to bring about a controlled change in volume through displacement of one of these regions to the other region.

In some embodiments, the hydraulic reservoir can be delimited or defined, at least in part, by a displaceable hydraulic stopper which is guided in a rigid, cylindrical side wall region. The hydraulic stopper is then arranged such that it can be displaced by the drive means, and may be directly accessible for the drive means.

In some embodiments, the hydraulic reservoir may be compressible as a whole, as was described for the product container. Also, the hydraulic reservoir can accordingly be designed such that the volume of the hydraulic reservoir can be altered without parts in contact with the hydraulic fluid sliding against one another. This leads to simplified manufacturing, because a sealed stopper can be omitted, and enables the hydraulic reservoir to take on a wide range of forms. For this, the hydraulic reservoir can also have at least one wall region which can be deformed or a surface that can be altered such that a change in volume of the hydraulic reservoir occurs. In some embodiments, the hydraulic reservoir has a wall region designed as bellows, a wall region made of a flexible film, or a wall region made of an elastomer material. In some preferred embodiments, the hydraulic reservoir has at least one or, in some preferred embodiments, two dimensionally stable end regions which can move against one another, e.g. in the form of a dimensionally stable end-side terminal wall. The volume of the hydraulic reservoir can be modified in various ways. It is thus possible to configure the administering device such that the hydraulic reservoir can be compressed by pressure. In some embodiments, it is possible to configure the administering device such that a first end region of the hydraulic reservoir can be displaced towards a second end region for a decrease in volume of the hydraulic reservoir. Alternatively or in addition, it is also possible for the first end region to be rotatable against the second end region for a change in volume of the hydraulic reservoir such that the hydraulic reservoir is virtually “wrung” or twisted for a decrease in volume (compression). The latter possibility represents a way to convert a rotating drive, as is available from usual motors, into translation of a stopper or an end region of the product container, without the need for a mechanical gearbox.

In some preferred emboidments, the hydraulic reservoir can be compressed as a whole, whereas the product container has a displaceable product stopper. In this case, the administering device can be used with standardized and tested ampoules, whereas the compressible hydraulic reservoir can be made and filled easily.

In some embodiments, to enable an easy filling level and function check, at least a partial region of an outer wall of the administering device is transparent or translucent. Checking is made easier if the hydraulic fluid is colored.

In one configuration, the housing has means for detachably fixing the drive unit such that the user can conveniently attach a drive unit to the device and can remove it after use. A plurality of appropriate means is conceivable. In one case, the means may comprise a recess for taking up a detent pawl of the drive unit.

The present invention also encompasses a system for administering a fluid product, which comprises an administering device according to the present invention and a compatible drive unit for detachable fastening on or in a drive-receiving region of the administering device. The drive unit is designed for supplying a driving pressure to the structure for hydraulic power transmission. In some preferred embodiments, this system is a “semi-disposable” system, i.e. only the actual administering device is designed as a disposable article, while the signficantly more expensive drive unit is reusable. The administering device may be, e.g., an infusion device for continuous administering of the product over a relatively long period or an injector for delivering single doses.

In some embodiments, the system is configured such that control of the administration rate of the fluid product takes place exclusively through control of the drive unit, whereby the administration rate is therefore not controlled on or by the hydraulic section. There are, therefore, no controllable valves or similar devices necessary, which would complicate manufacturing and increase the cost of the system.

In some preferred embodiments, an electric motor, e.g. a DC motor or step motor, is the drive means in the drive unit. For controlling the motor, electronic control means are provided, which may comprise a microcomputer or microprocessor as known per se. The drive unit further comprises a power source, e.g. in the form of one or more electric batteries, which can be disposable batteries or rechargeable batteries. Separate batteries for supplying the control means and motor can be provided to boost operational safety. Other possibilities also can be considered as power source, however. Accordingly, for example, it is conceivable to supply the motor with power inductively to be able to encapsulate the drive unit more easily. To transmit the drive power of the motor to the hydraulic reservoir, in one preferred configuration, the motor comprises an axially displaceable piston rod.

Whereas an arrangement with electromotor drive is suitable to administer the product continuously over a relatively long period, the system can however also be designed as an injector for single doses, administered once or at predetermined intervals. The drive unit can then be designed as an arrangement for manual administering of a predetermined dose of the fluid product. This arrangement can then be a purely mechanical arrangement without electric components. Such arrangements are known from commercially available injection pens.

In some embodiments, the system is configured such that “priming” takes place when the drive unit is attached to the administering device. The administering device and the drive unit are therefore designed such that when the drive unit is placed in the administering device, the structure for hydraulic power transmission is supplied with pressure such that initial delivery of the fluid product occurs when the product container is open.

In some embodiments, the present invention comprises a method of manufacturing an administering device, wherein the device is manufactured such that a hydraulic reservoir is filled only after a product container has been installed. The method therefore comprises the steps of providing a housing with the as yet unfilled hydraulic reservoir arranged therein and the product container filled with the fluid product, and subsequently filling the hydraulic reservoir with hydraulic fluid. This makes it possible to generate excess pressure in the product container already at the time of manufacture, such that “priming,” i.e. product delivery, automatically takes place when a catheter is being attached. For this, the hydraulic fluid in the hydraulic reservoir is supplied with excess pressure during filling.

In some preferred embodiments, filling the hydraulic reservoir happens via a membrane on the hydraulic reservoir, which can be punctured by a filling needle and closes again automatically after the needle is removed, e.g. a conventional septum. In some embodiments, the hydraulic reservoir is evacuated prior to filling through the needle, the membrane is punctured by a filling needle for filling, and the hydraulic fluid is then filled.

In some embodiments, the present invention comprises a method for operating a system comprising an administering device and a drive unit, wherein which the administration rate is controlled exclusively by the drive unit.

### BRIEF DESCRIPTION OF THE DRAWINGS

FIG. 1A shows a sectional view of an administering device according to one embodiment of the present invention prior to use with a separate drive unit;

FIG. 1B shows a sectional view of the administering device of FIG. 1A following installation of the drive unit;

FIG. 1C shows a sectional view of the administering device of FIG. 1A after use;

FIG. 2A shows a sectional view of an administering device according to another embodiment of the present invention prior to use with a separate drive unit;

FIG. 2B shows a sectional view of the administering device of FIG. 2A following installation of the drive unit;

FIG. 2C shows a sectional view of the administering device of FIG. 2A after use;

FIG. 3A shows a sectional view of an administering device according to another embodiment of the present invention prior to use with separate drive unit;

FIG. 3B shows a sectional view of the administering device of FIG. 3A following installation of the drive unit;

FIG. 3C shows a sectional view of the administering device of FIG. 3A after use; and

FIG. 4 shows a sectional view of an injector.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making the invention and/or its components may be selected from appropriate materials such as metal, metallic alloys, ceramics, plastics, etc. Generally, unless otherwise indicated, relative positional or orientational terms (e.g., upwardly, downwardly, above, below, etc.) are intended to be descriptive, not limiting.

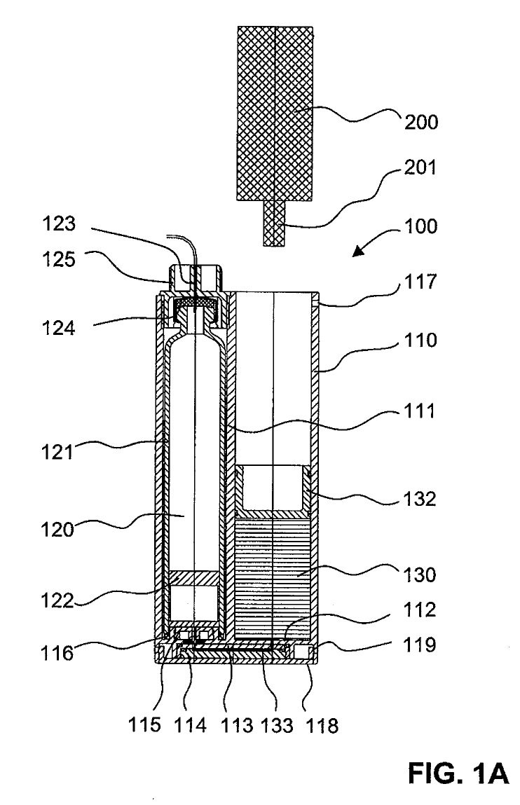

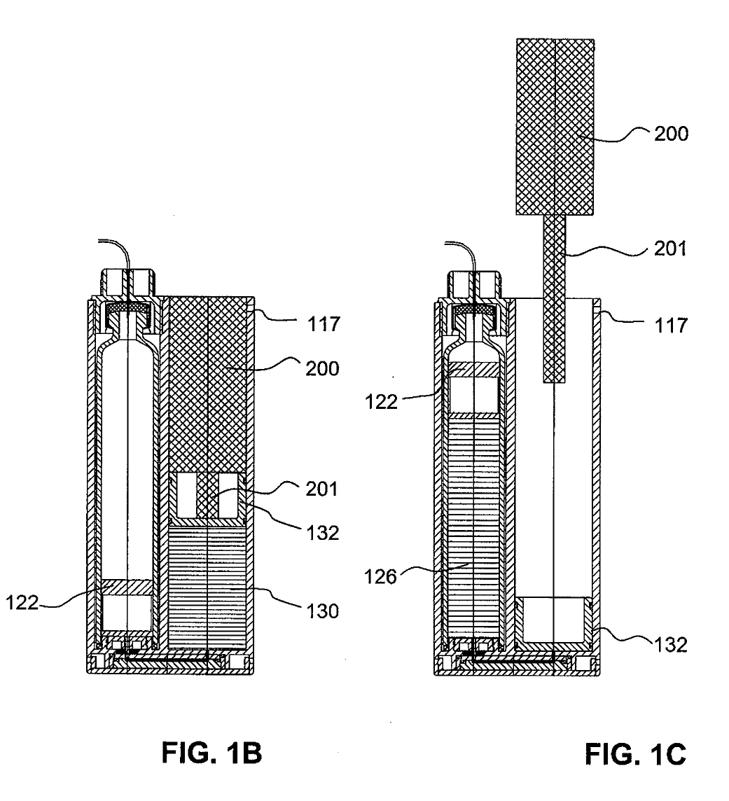

FIGS. 1A to 1C illustrate one embodiment of an administering device 100 according to the present invention together with a drive unit 200. These components or parts can be sold separately or jointly as a system. The administering device 100 and the drive unit 200 together form an infusion device or a medicament pump.

The administering device comprises a housing 110 which is subdivided by a partition 111 into a left and a right part. The partition 111 terminates downwardly in a housing bottom 112. In the left part, a receiving region for a product container is provided, in FIGS. 1A to 1C containing, for example, an ampoule 120 filled with a liquid medicament or therapeutic agent. The receiving region is a long cylindrical cavity. At the lower end of the cavity is an insert 115 which is held in an annular flange of the housing bottom 112 extending upwardly into the cavity and which rests on the housing bottom 112. In the right half of the housing, a cylindrical cavity is likewise provided, in which a stopper 132 (which also may be thought of and/or referred to as a hydraulic stopper) is guided displaceably and is sealed by two sealing rings which bear against the wall of the cavity. The stopper 132 and the wall of the cylindrical cavity jointly delimit or define a hydraulic reservoir 130, containing a hydraulic fluid. Between two flanges extending downwardly from the housing bottom 112, two inserts 113 and 114 are inserted successively from below, which jointly delimit or define a fluid channel 133 extending from the right to the left half of the housing. There is a fluid connection between the hydraulic reservoir 130 and the fluid channel 133 through an opening in the housing bottom. There is also a fluid connection on the left housing side from the fluid channel 133 through the insert 115 to the receiving region for the ampoule 120. In this way, there is a continuous fluid connection (which also may be thought of and/or referred to as a hydraulic section) between the hydraulic reservoir 130 and the receiving region for the ampoule 120. The housing 110 is closed downwardly by a lower cover 118 which is latched by detent lugs 119 in corresponding openings of the housing 110.

A conventional glass ampoule 120 is inserted in the receiving region. The ampolule is sealed at the bottom by a displaceable stopper 122 (which also may be thought of and/or referred to as a product stopper), which is guided by or in the cylindrical outer wall 121 of the ampoule. At the upper end, the ampoule is sealed by a conventional cover 124 with a septum. The lower, open edge region of the outer wall 121 of the ampoule projects into an annular space between the wall of the housing 110 and the annular flange holding the insert 115, and lies there on a seal in the form of a crimped seal 116 with a square cross-section. At the upper end, the ampoule is held by a closure 125 simultaneously acting as a connecting adapter which is screwed or clicked into the upper edge region of the receiving region of the housing 110. The closure 125 holds a hollow needle 123 which penetrates the septum of the ampoule cover 124 and accordingly forms an opening of the product container. A conventional catheter joins the hollow needle 123, but instead of a catheter an injection needle could be directly present.

On the right housing side is a receiving region for a drive unit 200 which is illustrated schematically. This receiving region is limited downwardly by the stopper 132 which seals off the hydraulic reservoir 130 to the top. The situation directly after insertion of the drive unit 200 in the receiving region is illustrated in FIG. 1B. The drive unit 200 is held by appropriate means, e.g. a detent pawl, not illustrated, in a recess 117 of the outer housing wall. The hydraulic stopper 132 has on its top side a recess, into which a piston rod 201, likewise illustrated schematically, projects after insertion of the drive unit 200.

The piston rod 201 can be extended axially downwardly by appropriate drive means controlled in the drive unit 200. An electromotor, e.g. a DC motor or a stepper motor, which drives the piston rod via an appropriate gearbox may comprise the drive means. For this purpose, the piston rod 201 e.g. can usually be designed as a threaded rod, on which a drive nut runs that is driven by the motor (not illustrated here).

To dispense a medicament or therapeutic agent contained in the ampoule 120 through the hollow needle 113, the motor of the drive unit 200 is set in motion. It now gradually drives the piston rod 201 down. In so doing, the hydraulic stopper 132 is pressed down. The hydraulic fluid is pressed through the fluid channel 133 into the receiving region for the ampoule 120 by the resulting pressure in the hydraulic reservoir. Here it exerts an upwardly acting force on the product stopper 122 in the ampoule 120, whereby pressure builds in the ampoule 120, by which the liquid medicament or agent contained in the ampoule is discharged through the hollow needle 113. In other words, displacement of the piston rod 121 leads to displacement of the hydraulic stopper 132, which in turn leads to displacement of the product stopper 122 in the ampoule 120 by way of the hydraulic section. Between the insert 115 and the product stopper 122 a reservoir 126 is formed by this displacement, which accepts the hydraulic fluid exiting from the fluid connection 133 and originating from the hydraulic reservoir. This shift reservoir 126 is evident in FIG. 1C, which illustrates the situation after the hydraulic stopper 132 has been pressed fully downwardly, the hydraulic reservoir 130 has therefore been fully emptied, and after the drive unit 200 has again been removed from the housing 110. The shift reservoir 126 is limited here to the side by the circumferential side wall 121 of the ampoule 120 and to the top by the side of the product stopper 122 facing away from the medicament or agent. When the piston rod 201 is extended out of the drive unit 200 the volume of the hydraulic reservoir 130 therefore drops, in that the hydraulic fluid flows through the fluid connection into the shift reservoir 126, and the volume of the shift reservoir 126 increases to the same extent.

The administration rate, therefore the quantity of the discharged substance per time unit, is controlled in the process by the operation and/or control of the motor in the drive unit 200. The administration rate is therefore not determined by preset properties of the fluid connection, such as its length or cross-section, but can be controlled specifically by the drive unit 200. The cross-section of the fluid connection is sufficiently large throughout for the properties of the fluid connection for controlling the administration rate to make no or only a negligible contribution. In some preferred embodiments, there is no flow-limiting constriction anywhere in the fluid connection. Any preferred administration rate can thus be set in meaningful limits via the drive unit, without this rate being limited by the fluid connection. The fluid connection therefore serves just as power transmission from the drive unit to the product container, therefore acting, one might say, as a “liquid piston rod”.

In one case, control takes place by manual switching on and off of the motor. In some preferred embodiments, the motor can be controlled electronically, e.g. by a suitable microcomputer or microprocessor. This allows individual adaptation of the administration rate to the needs of the patient. For example, a basal rate for continuous administering can freely be set, selected or programmed in this way. It is also possible to control the motor specifically such that it discharges a predetermined bolus on request. Therefore, for example, it is possible that to administer the basal rate the motor is moved at adjustable time intervals in the range of seconds to minutes in each case by a fixed amount, e.g. a drive nut is rotated about a fixed angle. Control then is easily carried out by selecting the time intervals. For a bolus the motor is then additionally moved by a further selectable amount. Instead of this, it is also possible to regulate the motor such that by request of the user (e.g. on actuation of a corresponding key or switch) in each case a single dose is dispensed. The drive unit will also as normally include a power source, e.g. an electric battery (disposable battery or rechargeable battery). It may also be fitted with a display on which e.g. the adjusted rate and/or other operating data can be displayed. In addition, there can be control elements such as e.g. switches, keys or dials.

The administering device 100 can be manufactured very cost-effectively. All necessary parts, perhaps including the ampoule, can be made from plastic via injection molding. As a result, the administering device is suited for use as a disposable article which is thrown away, together with the ampoule and the catheter, after the ampoule has been emptied once. The drive unit 200 on the other hand, which can easily be removed from the administering device 100, can be used repeatedly. The system comprising the administering device 100 and the drive unit 200 can therefore be designated as a “semi-disposable” system, i.e. only the conveniently, inexpensively made part is thrown away, while the more expensive components are used repeatedly. Compared to a system in which only the ampoule is exchanged, the system in accordance with the present invention has a number of advantages, e.g. the exchange of the administering device is very much easier and less critical in terms of hygiene and therefore requires less time and less training. A further advantage is that the same drive unit can be used for several different ampoule sizes, since administering devices for different ampoule sizes, though with the same size for receiving the drive unit, can readily be manufactured. Conventional medicament and/or therapeutic agent ampoules can be used as ampoules, such as standard ampoules with 1.5 ml, 2 ml or 3 ml capacity. If the medicament or substance to be delivered is an insulin solution, then an ampoule of 1.5 ml capacity is usually administered over a period of a few days, e.g. 3 days, or an ampoule of 3 ml capacity is administered over a period of 1 week. Due to its construction the administering device can be kept very compact. It is thus possible, for example, to manufacture the device with inserted drive unit and a standard ampoule of 3 ml capacity with a thickness of less than 15 mm.

The present invention encompasses embodiments of a method of making administering devices, e.g. administering device 100, wherein the stopper 132 and the inserts 113, 114 and 115 are placed in the housing. The pre-filled ampoule 120 is likewise placed in the housing and fixed with the closure 125. The hydraulic fluid is injected. This can be done e.g. via a conduit, not illustrated, which terminates in the fluid channel 133 and is suitably sealed to the outside, e.g. by a septum or a one-way valve. Before that, in some embodiments, the air contained in the fluid channel 133 and possibly in the hydraulic reservoir 130 may be suctioned out to prevent air bubbles from forming in the hydraulic fluid. On completion of the filling procedure, the fluid can be placed under certain excess pressure, wherein, in this case, the movement of the hydraulic stopper 132 to the top is limited by a stop, not illustrated here. Because of this, certain excess pressure also arises in the ampoule 120 such that a small quantity of the substance to be dispensed is pressed through the hollow needle and into the catheter as soon as the septum pierces the cover 124. This results in venting of the catheter (“priming”). At the same time, the product stopper 122 is moved initially relative to the outer wall 121 of the ampoule 120 such that initial jamming is prevented during subsequent administering of the medicament.

In some embodiments, it is also possible to insert the ampoule after filling of the hydraulic reservoir. In this case, the fluid connection is first closed off to the receiving region for the product container, e.g. by a septum, and the insert 115 is designed such that it pierces this septum when the ampoule is inserted, e.g. with a hollow needle arranged on the insert. When the hydraulic fluid was filled under pressure, this pressure is transferred from this point on to the ampoule in turn, enabling automatic “priming.”

In addition or instead the drive unit 200 can be designed such that it exerts a force on the hydraulic stopper 132 when inserted into the housing 110, whereby the stopper is moved slightly downwardly. Assuming that the catheter was previously attached by the patient to the ampoule this also results in “priming,” the initial displacement of the product stopper and initial ejection of a certain quantity of the medicament for venting.

In some preferred embodiments, to avoid fluctuations in air pressure from influencing the administration rate of the product, the drive unit 200 is arranged on the housing such that fluctuations in air pressure cannot act on the hydraulic stopper 132. For instance, the drive unit can be sealed against the housing such that during operation there is permanent subpressure between drive unit and hydraulic stopper, resulting in the hydraulic stopper 132 being pressed permanently against the piston rod 201. It is also possible to place the piston rod detachably on the hydraulic stopper 132 such that during operation it cannot move away from the piston rod. For this purpose, a positive and/or non-positive connection between the hydraulic stopper and the piston rod can be provided “under tension” along its lengthwise direction. In some embodiments, it is possible to provide a closure which can be locked and unlocked by relative rotation of piston rod and hydraulic stopper. For this purpose, a holding element can be designed on the hydraulic stopper, in which a corresponding holding element can engage on the piston rod through rotation such that undercuts of both holding elements prevent axial separation. The closure can, for example, be a bayonet connection. In this case, one holding element may have an axial longitudinal slot, to the end of which a short transverse slot joins at a right angle. The other holding element has a knob-like boss which is inserted into the transverse slot and thereby causes a positive connection between the holding elements in an axial direction. An example of a non-positive connection is a suitable magnetic connection. A solid connection between piston rod and hydraulic stopper can thus be easily made such that an administering device according to the present invention can easily ensure that the administration rate is not influenced by air pressure. Other suitable positive and non-positive connection are possible.

To make it easy to read the filling level of the medicament or therapuetic agent or of the hydraulic fluid a region of the outer wall of the housing 110 can be transparent or translucent, and/or could be provided with a scale or indicia. A region of the outer wall of the housing bordering the hydraulic reservoir 130, via which the filling level of the hydraulic fluid can be read, may be well-suited for this purpose. To make reading easier or to make leaks more evident, the hydraulic fluid can be colored. Leakage from the stopper associated with the ampoule, which might lead to hydraulic fluid entering the ampoule, could be easily be recognized in this way.

An appropriate incompressible fluid is used as hydraulic fluid. For example, deionized or distilled water could be used, which is harmless should the stopper of the medicament ampoule lose its seal and cause small quantities of the hydraulic fluid to enter the ampoule. However, other fluids are also suitable, e.g. oils. With use of highly viscous fluids such as castor oil, glycerine or silicone oil the cross-section of the fluid connection should be sufficiently large not to limit the throughflow rate during operation. In this case, the minimal size of the fluid connection transverse to the throughflow direction (minimal diameter for a pipe) should be e.g. at least 1 millimeter. With use of fluids of lesser viscosity, such as water, however, lesser physical dimensions are also possible.

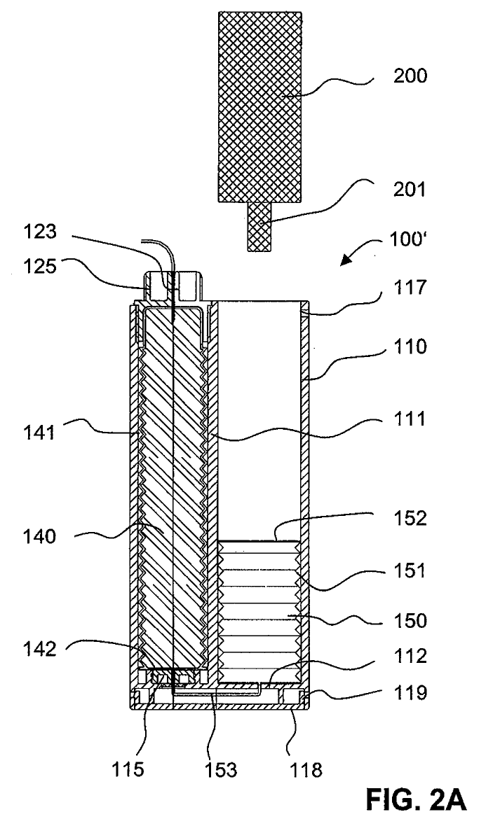

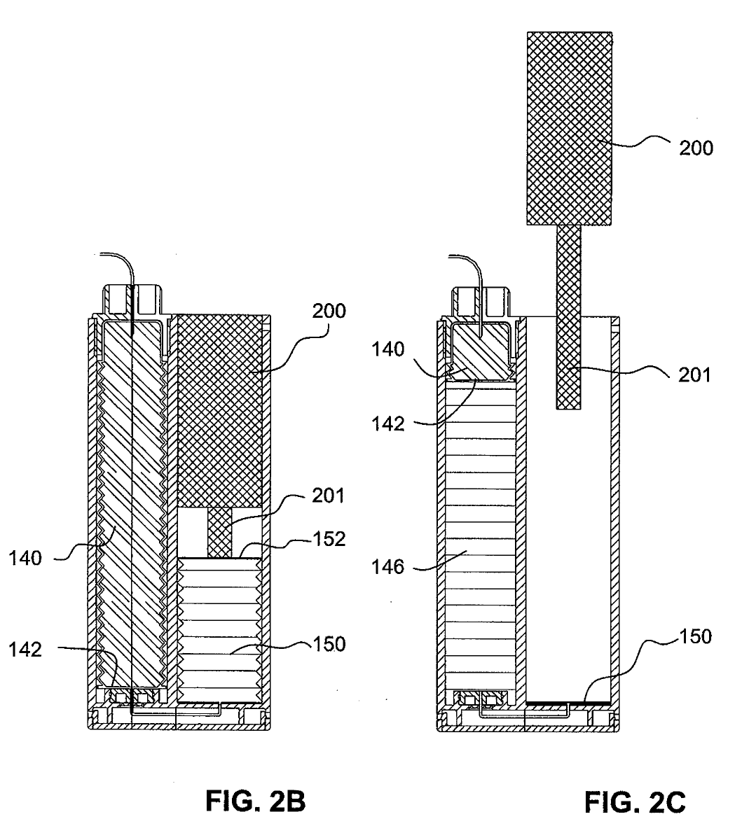

FIGS. 2A to 2C show a second embodiment of the present invention. Similar parts are designated by the same reference numerals as in FIGS. 1A to 1C. This embodiment differs from the embodiment of FIGS. 1A to 1C in that the product container 140 and the hydraulic reservoir 150 each are compressible as a whole. For this purpose, the circumferential side wall 141 of the product container is designed as bellows, i.e. it has a plurality of crease lines along which adjacent side wall regions can be folded onto one another. Similarly, the hydraulic reservoir 150 also has a side wall 151 designed as bellows. By the product container 140 and the hydraulic reservoir 150 being compressible as a whole, the need to seal a displaceable element such as the hydraulic stopper or the product stopper of the preceding embodiment such that no fluid can escape does not apply. The administering device can be produced more easily and cost-effectively. A further difference from the embodiment of FIGS. 1A to 1C is that the fluid connection is made in another way, here by a conduit 153 arranged between the housing bottom 112 and the lower cover 118. Again this conduit has a sufficiently large cross-section to not contribute significantly to controlling the administration rate. A conduit could be omitted entirely. In this case, the fluid connection is formed by the cavity between housing bottom 112 and lower cover 118 or a channel correspondingly designed in the lower cover 118, wherein the lower cover 118 is then sealed against the housing 110.

FIG. 2A illustrates the device prior to insertion of the drive unit 200; FIG. 2B immediately after insertion. The drive unit presses during operation on the upper, dimensionally stable limit 152 of the hydraulic reservoir 150 and compresses the latter accordingly. Because of this, hydraulic fluid is pressed through the conduit 153 against the (here dimensionally stable) bottom 142 of the product container 140, whereby the latter is likewise compressed. The hydraulic fluid collects in the region between the lower limit of the receiving region for the product container and the bottom of the product container 140, wherein this region forms a shift reservoir 146. This is evident from FIG. 2C, which shows the device after the hydraulic reservoir has been fully compressed and the drive unit 200 removed.

The hydraulic reservoir and/or the product container can also be designed other ways, too, e.g. with a wall made of elastic rubber or another elastomer or a flexible, but inelastic, film. The insert 115 may be omitted without replacement; the conduit 153 could then terminate in the receiving region for the product container directly through the housing bottom 112. In the illustrated embodiment, wherein the shift reservoir is limited by the housing wall and the product container, an appropriate independent container of variable volume could be present in the receiving region as a closed shift reservoir which attaches to the fluid connection, here the conduit 153. This applies equally for the first embodiment. During filling, the hydraulic reservoir can be placed under excess pressure to enable automatic “priming,” wherein the elastic properties of the hydraulic reservoir can farther contribute to maintaining the excess pressure over a relatively long period.

In the embodiment of FIGS. 2A to 2C, the hydraulic reservoir is compressed by being pressed together. Instead of this, it is also possible that the hydraulic reservoir is compressed (therefore its volume decreasing) by the upper end of the hydraulic reservoir being rotated against the lower end. As a result, the capacity of the reservoir is, one might say, “wrung out.” For this purpose, there can be a more general drive shaft instead of the piston rod 201, which can be driven by the drive unit to rotate. At the upper end of the hydraulic reservoir there can be a connector, with which a complementarily designed end of the drive shaft can be engaged such that a solid connection results in the direction of rotation, e.g. in the form of a rotational stop.

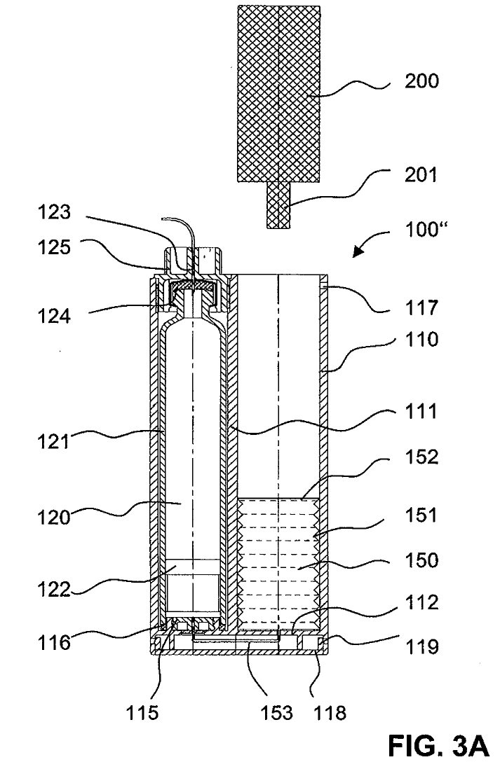

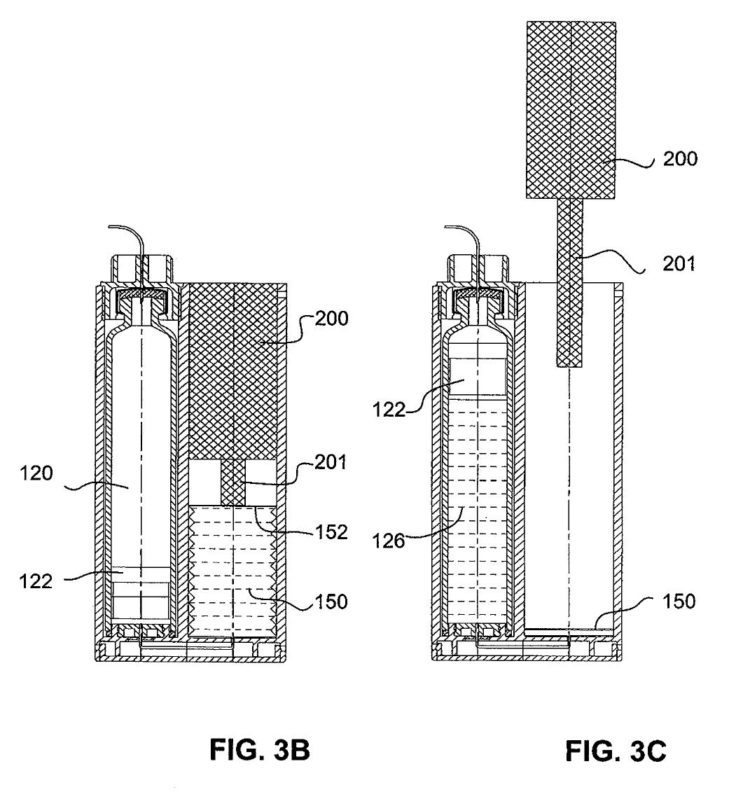

Another embodiment of an administering device 100″ is illustrated in FIGS. 3A to 3C. Here an ampoule with displaceable stopper is combined with a hydraulic reservoir compressible as a whole. For further details, reference is made to the implementations for the first and second embodiments above. This arrangement combines the simple and cost-effective manufacturing of a compressible, bellows-like hydraulic reservoir, as well as the possibility of easily providing the latter with excess pressure, with the proven qualities of a conventional ampoule. It is also possible, however, to combine a product container compressible as a whole with a hydraulic reservoir which is limited by a movable hydraulic stopper.

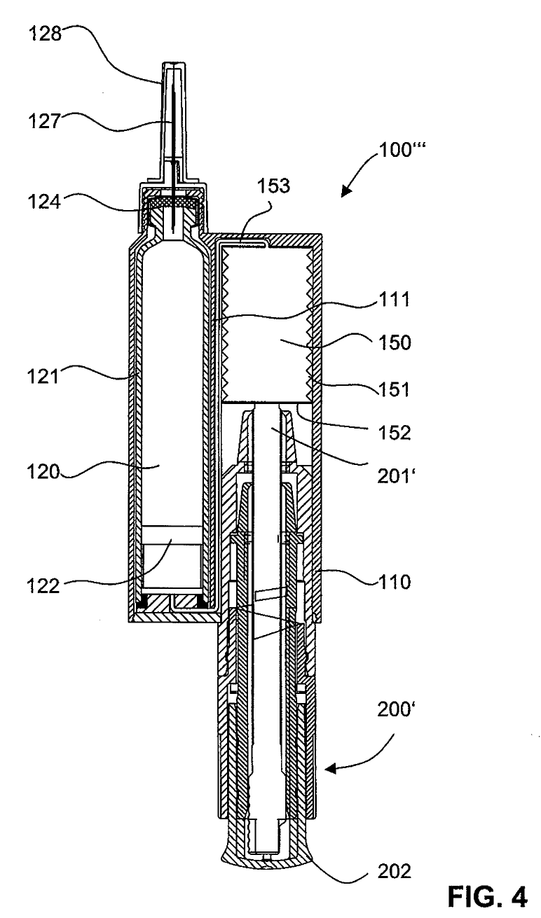

FIG. 4 shows another embodiment in which the administering device 100′″ serves as injector for single doses. Again, the administering device has a housing 110 which is divided by a partition 111 into a left and right part. In the left part, an ampoule 120 with displaceable stopper 122 is arranged. Instead of an ampoule, a compressible as a whole product container can also be provided. An injection needle 127, covered with a protective cap 128, is arranged on the closure 125, instead of a catheter. The right housing part is open downwardly. In this part, a hydraulic reservoir 150 is arranged, illustrated here for example as a compressible container with bellows-like side wall 151 and dimensionally stable lower limit 152. It could be designed differently, e.g. as a cylinder with a displaceable stopper or as a container with a flexible side wall made of a film. From the hydraulic reservoir 150 a fluid connection in the form of a conduit 153 leads to the receiving region for the ampoule, wherein the conduit 153 runs partially in the partition 111. For other possible configurations reference is made to the illustration of the preceding embodiments.

A drive unit 200′, here designed as a mechanical arrangement for manual administering of a predetermined dose of the fluid product, is set in the right housing part from below. For this purpose, the drive unit comprises the drive components of an injection pen known per se. Through pressure on the push button 202, the drive unit generates a predetermined, adjustable, one-off advance motion of a piston rod 201′. Because of this, the hydraulic reservoir 150 is compressed, resulting in the medicament being delivered through the injection needle 127. The advance of the piston rod 201′ and thus the discharged quantity can be pre-selected by rotating the push button 202 or a dosing ring and read on a scale on the knob or on the housing. Suitable configurations of the drive components are known from injectors of the prior art in a wide range of forms. WO 97/17096 A1 and DE 103 43 548 A1 disclose injectors powered in this way, in which a driven element corresponding to the piston rod 201′ can be retracted after a complete emptying. The drive unit of such an injector is well-suited for a “semi-disposable” design, since the drive unit can be reused a number of times. DE 199 00 792 C1 discloses an example of an injector, the drive unit of which is suitable for a disposable product which is thrown away completely after total emptying of the product container.

Again, the cross-section of the fluid connection is made large enough for the fluid connection not to substantially influence the rate with which the medicament is expelled through the injection needle 127, therefore not opposing the advance of the piston rod 201′ by any appreciable resistance. The administration rate is thus again controlled substantially exclusively by the drive unit, here triggered by manual pressure on the push button 202.

Of course, a plurality of other configurations is possible, and the invention is not limited to the examples described here. The hydraulics thus enable a large number of structural forms, e.g. structural forms in which the product container and the hydraulic reservoir are arranged successively along the axis of the product container instead of adjacently. It is also possible that the hydraulic reservoir encloses the product container radially as an annular space. In this case, the hydraulic reservoir can be limited or defined in part by a displaceable ring piston. It is also conceivable that the direction in which the hydraulic stopper is moved or the hydraulic reservoir is compressed, and the direction in which the product stopper is moved or the product container is compressed, are not parallel, but enclose an angle, e.g. are perpendicular to one another. There are, therefore, a multiplicity of structural forms possible for adapting the administering device to selected requirements.

In some embodiments, the drive unit does not need to be inserted fully into the housing of the administering device, as in the above three embodiments, but can also be attached to the housing, e.g. to the side. In other words, the drive-receiving region does not necessarily have to be a cavity.

Means for limiting throughflow can be provided at the outlet of the product container to the catheter or to the injection needle, e.g. in the form of a constriction or a valve. Using a valve can also ensure that the product is not administered unintentionally at an undesired point in time. In this respect, a valve offers additional safety. The administration rate can also be controlled alone or additionally via the valve.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An administering device for administering a fluid product with a regulatable administration rate, comprising a housing comprising:
a drive-receiving region for a drive unit;
a product-receiving region for receiving a product container containing the fluid product; and
a structure for hydraulic power transmission between the drive-receiving region and the product-receiving region, wherein the structure for hydraulic power transmission makes no contribution to regulating the administration rate.

2. The administering device as claimed in claim 1, wherein the structure for hydraulic power transmission comprises:
a hydraulic reservoir containing a hydraulic fluid, wherein the hydraulic reservoir is supplied with a driving pressure by a drive unit operably associated with the drive-receiving region; and
a fluid connection between the hydraulic reservoir and a shift reservoir, wherein the fluid connection and the shift reservoir transmit the driving pressure in the hydraulic reservoir to the fluid product in the product container arranged in the product-receiving region, and wherein the fluid connection has a cross-section sufficient that the fluid connection has a negligible effect on the administration rate.

3. The administering device as claimed in claim 2, wherein the product container comprises a cylindrical side wall region and a product stopper displaceable in the container by expansion of the shift reservoir.

4. The administering device as claimed in claim 3, wherein the shift reservoir is delimited at least partially by the cylindrical side wall region and the side of the product stopper away from the fluid product.

5. The administering device as claimed in claim 2, wherein the product container is compressible.

6. The administering device as claimed in claim 5, wherein the product container comprises at least one wall region having at least one of a variable form and dimension.

7. The administering device as claimed in claim 2, wherein the hydraulic reservoir comprises a cylindrical side wall region and a hydraulic stopper guided displaceably in resrevoir, the stopper displaceable by the drive unit.

8. The administering device as claimed in claim 2, wherein the hydraulic reservoir is compressible.

9. The administering device as claimed in claim 8, wherein the hydraulic reservoir has at least one wall region having at least one of a variable form and dimension.

10. The administering device as claimed in claim 2, wherein at least a partial region of an outer wall of the administering device is transparent or translucent.

11. The administering device as claimed in claim 10, wherein the hydraulic fluid is colored.

12. The administering device as claimed in claim 2, wherein the housing comprises means for detachably coupling the drive unit.

13. A system for administering a fluid product, comprising an administering device having a controllable associated administration rate and comprising a housing comprising a drive-receiving region for a drive unit, a product-receiving region for a product container containing the fluid product, a structure for hydraulic power transmission between the drive-receiving region and the product-receiving region, wherein the structure for hydraulic power transmission makes no contribution to controlling the administration rate, and a drive unit for coupling to the drive-receiving region, the drive unit adapted to supply the structure for hydraulic power transmission with a driving pressure.

14. The system as claimed in claim 13, wherein the system is configured such that control of the administration rate takes place exclusively by the drive unit.

15. The system as claimed in claim 14, wherein the drive unit comprises an electric motor.

16. The system as claimed in claim 15, wherein the drive unit further comprises an electronic control means for controlling the motor.

17. The system as claimed in claim 16, wherein the drive unit further comprises a piston rod axially displaceable by the motor, the piston rod adapted to act on the structure for hydraulic power transmission.

18. The system as claimed in claim 13, wherein the drive unit provides for manual administering of a predetermined dose of the fluid product.

19. The system as claimed in claim 13, wherein the administering device and the drive unit are designed such that when the drive unit is operably coupled to the administering device the structure for hydraulic power transmission is supplied with pressure such that an initial delivery of the fluid product occurs when the product container is open.

20. A method for operating a system comprising an administering device having a controllable associated administration rate and comprising a housing comprising a drive-receiving region for a drive unit, a product-receiving region for a product container containing the fluid product, a structure for hydraulic power transmission between the drive-receiving region and the product-receiving region, wherein the structure for hydraulic power transmission makes no contribution to controlling the administration rate, and a drive unit for coupling to the drive-receiving region, the drive unit adapted to supply the structure for hydraulic power transmission with a driving pressure, wherein the administration rate is controlled exclusively by the drive unit.

21. A method for manufacturing an administering device having a controllable associated administration rate for administering a fluid product and comprising a housing comprising a drive-receiving region for a drive unit, a product-receiving region for receiving a product container containing the fluid product, a structure for hydraulic power transmission between the drive-receiving region and the product-receiving region, wherein the structure for hydraulic power transmission makes no contribution to controlling the administration rate, the method comprising the steps of:
providing the housing with an as yet unfilled hydraulic reservoir arranged therein and the product container filled with the fluid product; and
subsequently filling the hydraulic reservoir with a hydraulic fluid.

22. The method as claimed in claim 21, wherein the hydraulic fluid in the hydraulic reservoir is supplied with excess pressure during filling.
